(19) United States
(12) Patent Application Publication
(10) Pub. No.: US 2020/0171076 A1
(43) Pub. Date: Jun. 4, 2020

(54) SEVELAMER FOR THE TREATMENT AND/OR PREVENTION OF AORTIC STENOSIS

(71) Applicant: QUEEN MARY UNIVERSITY OF LONDON, Greater London (GB)

(72) Inventor: David Samuel WALD, London (GB)

(21) Appl. No.: 16/621,463

(22) PCT Filed: Jun. 7, 2018
(86) PCT No.: PCT/GB2018/051556

§ 371 Date: Dec. 11, 2019

(30) Foreign Application Priority Data
Jun. 12, 2017 (GB) 1709337.8

(51) Int. Cl. A61K 31/785; A61K 31/663

(52) U.S. Cl. CPC A61K 31/785; A61K 9/0053; A61K 31/663

(57) ABSTRACT

A method of treating and/or preventing aortic stenosis in a patient in need thereof comprises administering to the patient a therapeutically effective amount of sevelamer or a pharmaceutically acceptable derivative, mixture or salt thereof (e.g. sevelamer hydrochloride or sevelamer carbonate). The method may attenuate or arrest the deposition of calcium phosphate crystals on the aortic valve of the patient. The method is applicable to patients who do not have hyperphosphatemia, including those having a serum phosphate level of 2.5 mg/d L to 4.5 mg/d L.

### FIELD OF THE INVENTION

The present invention relates to methods of treating and/or preventing aortic stenosis in a patient. It also relates to a method of identifying patients that would benefit from said treatment.

### BACKGROUND TO THE INVENTION

Aortic stenosis is a serious heart valve disorder affecting about 12% of people over age 75, about one quarter of whom are severely affected. It is caused by progressive deposition of calcium phosphate crystals on the aortic valve (aortic sclerosis), leading to obstruction of blood flow from the heart. Mild aortic stenosis becomes severe within about 5 years. Progressive valve narrowing (stenosis) can lead to symptoms, such as chest pain, syncope or heart failure. Death follows symptoms of cardiac insufficiency in most cases unless the valve is surgically replaced. About 1.3 million people over age 75 have aortic stenosis in the UK, and this number is expected to double over the next few decades as the population ages. Based on current prevalence estimates in people over age 75, there are approximately 4.8 million people in the European Union and 2.5 million in the USA with aortic stenosis.

The estimated 5-year survival of aortic stenosis patients is about 40% in the absence of aortic valve replacement surgery, which is the only treatment available. About 67,500 surgical aortic valve replacements are carried out in the USA each year, and it is the most common valve surgery performed. Most aortic valve replacements are done by open heart surgery. This costs about £19,000 per procedure and is complicated by death, stroke or bleeding in about 5%. For some patients the risk of surgery is too high. Transcatheter aortic valve implantation is an alternative method using a catheter (thin tube) and the arterial system to access the heart. This alternative method carries a complication rate of 5-10%, and a cost of about £25,000.

Currently, there is no known means of preventing the progression of aortic stenosis. Management consists of “watchful waiting”—periodic assessments over several years and emergency valve replacement when symptoms develop. About 1 in 10 patients with aortic stenosis die suddenly, and in those who undergo surgery there is about a 1 in 20 chance of serious complications (including stroke and death). It would therefore be desirable to find new therapies for treating or preventing progression of aortic stenosis.

### SUMMARY OF THE INVENTION

It has now been found that sevelamer may prevent aortic stenosis by reducing the deposition of calcium phosphate crystals on the aortic valve of the patient and slowing or arresting valve stenosis.

The present invention provides, in a first aspect, a method of treating and/or preventing aortic stenosis in a patient in need thereof, said method comprising administering to the patient an effective amount of sevelamer or a pharmaceutically acceptable derivative, mixture or salt thereof.

The present invention also provides, in a second aspect, a method of identifying a subject that would benefit from treatment with sevelamer or a pharmaceutically acceptable derivative, mixture or salt thereof, said method comprising:

(a) determining whether the subject shows a symptom of aortic stenosis; and

(b) where the subject shows a symptom of aortic stenosis, making a record that the subject would benefit from treatment with sevelamer or a pharmaceutically acceptable derivative, mixture or salt thereof.

The present invention also provides, in a third aspect, a method of treating and/or preventing aortic stenosis in a patient comprising:

(a) determining whether the patient has a symptom of aortic stenosis; and

(b) where the subject shows a symptom of aortic stenosis, administering to the patient an effective amount of sevelamer or a pharmaceutically acceptable derivative, mixture or salt thereof.

Embodiments of the first, second and third aspects include the following:
(i) the method attenuates or arrests the deposition of calcium phosphate crystals on the aortic valve of the patient;
(ii) the patient does not have hyperphosphatemia;
(iii) the patient's serum phosphate level is 2.5-4.5 mg/dL;
(iv) the patient does not have chronic kidney disease;
(v) the sevelamer salt is sevelamer hydrochloride or sevelamer carbonate;
(vi) the sevelamer or a pharmaceutically acceptable derivative, mixture or salt thereof is administered orally, preferably with or immediately after meals;
(vii) 800 mg of sevelamer is administered three times daily;
(viii) about 1-14 g of sevelamer is administered per day;
(ix) about 2-8 g of sevelamer is administered per day;
(x) about 2 g, about 3 g, about 4 g, about 5 g, about 6 g, about 7 g, or about 8 g of sevelamer is administered per day;
(xi) the sevelamer is co-administered with a bisphosphonate;
(xii) the patient has been identified as having aortic stenosis or being at risk of developing aortic stenosis;
(xiii) sevelamer or a pharmaceutically acceptable derivative, mixture or salt thereof reduces serum total cholesterol and LDL cholesterol;
(xiv) sevelamer or a pharmaceutically acceptable derivative, mixture or salt thereof may reduce the risk of heart attack and/or stroke; and
(xv) the screening of subjects to identify those that show symptoms of aortic stenosis involves echocardiography and/or a Computed Tomography (CT) scan.

For the avoidance of doubt, the embodiments described herein can be combined unless context clearly dictates otherwise. Furthermore, the first, second and third aspects, and the embodiments thereof can be combined with other optional features of the present invention as disclosed elsewhere herein unless context clearly dictates otherwise.

### BRIEF DESCRIPTION OF THE FIGURES

FIG. 1 shows the chemical structure of sevelamer, in which: a, b=number of primary amine groups and a+b=9, c=number of crosslinking groups and c=1, and m=large number to indicate extended polymer network.

FIG. 2 shows a flow chart of the 18-week cross-over trial in patients with mild-moderate aortic stenosis.

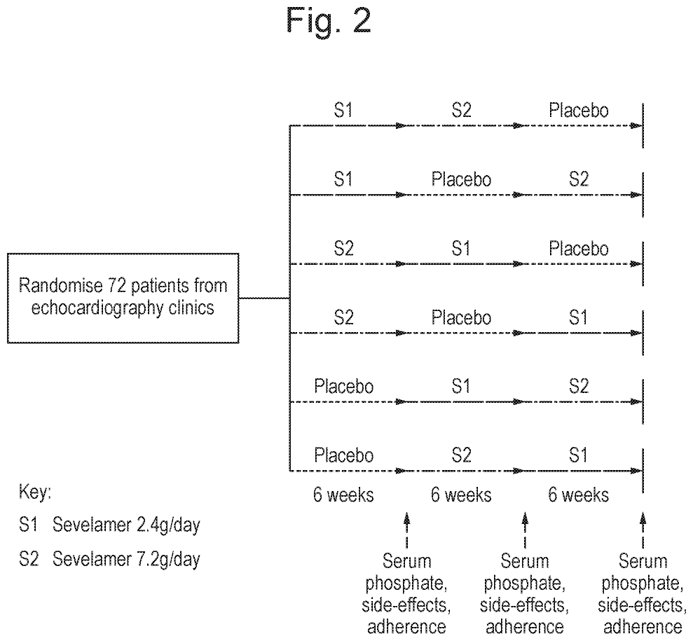

### DETAILED DESCRIPTION

The present invention provides a method of treating and/or preventing aortic stenosis comprising administering to a patient an effective amount of sevelamer or a pharmaceutically acceptable derivative, mixture or salt thereof. In some embodiments, the method attenuates the deposition of calcium phosphate crystals on the aortic valve of the patient. In other embodiments, the method arrests the deposition of calcium phosphate crystals on the aortic valve of the patient.

The present invention also provides a method of treating and/or preventing aortic stenosis in a patient comprising: (a) determining whether the patient has a symptom of aortic stenosis; and (b) where the subject shows a symptom of aortic stenosis, administering to the patient an effective amount of sevelamer or a pharmaceutically acceptable derivative, mixture or salt thereof. The determining in step (a) may involve echocardiography and/or a Computed Tomography (CT) scan.

The present invention also provides a method of identifying a subject that would benefit from treatment with sevelamer or a pharmaceutically acceptable derivative, mixture or salt thereof, said method comprising: (a) determining whether the subject shows a symptom of aortic stenosis; and (b) where the subject shows a symptom of aortic stenosis, making a record that the subject would benefit from treatment with sevelamer or a pharmaceutically acceptable derivative, mixture or salt thereof. The determining in step (a) may involve echocardiography and/or a Computed Tomography (CT) scan. The making of a record according to step (b) may comprise data input into a database, e.g. on a computer or on a paper record system. The record may be in the form of a prescription, e.g. a prescription for sevelamer or a pharmaceutically acceptable derivative, mixture or salt thereof.

### Sevelamer

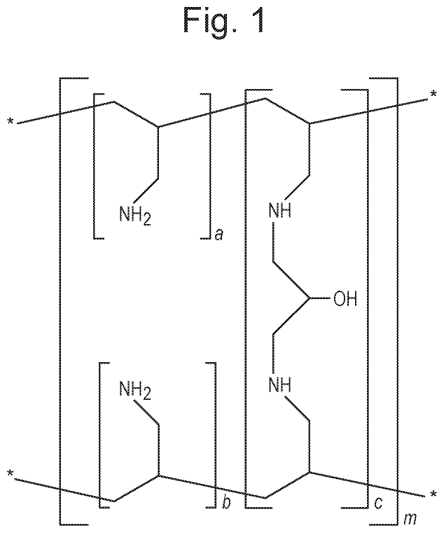

Sevelamer (see FIG. 1) is a metal-free, non-absorbed polymeric anion exchange resin, which binds to dietary phosphate. It consists of polyallylamine that is crosslinked with epichlorohydrin. Several phosphate-binding drugs are available, including sevelamer, lanthanum, calcium-acetate, aluminium and sucroferric oxyhydroxide. However, sevelamer is the most widely used. Two formulations exist: sevelamer hydrochloride and sevelamer carbonate, but each formulation has been shown to lower serum phosphate to the same extent. Sevelamer is marketed by Sanofi under the trade names Renagel (sevelamer hydrochloride) and Renvela (sevelamer carbonate).

Sevelamer is used to treat hyperphosphatemia in patients with chronic kidney disease. When taken with meals, it binds to dietary phosphate and prevents its absorption. Sevelamer has been used to lower phosphate in patients with end-stage renal failure, who have very high levels (2-3× physiological) of phosphate due to a failure of renal excretion. Sevelamer has also been found to reduce calcium phosphate levels. Thus, sevelamer may prevent aortic stenosis by reducing calcium phosphate deposition and slowing or arresting valve stenosis.

In some embodiments, sevelamer or a pharmaceutically acceptable derivative, mixture or salt thereof is co-administered with bisphosphonates. Bisphosphonates lower serum calcium, and may complement the phosphate-lowering activity of sevelamer or a pharmaceutically acceptable derivative, mixture or salt thereof.

In the prior art, it was not known whether sevelamer lowers serum phosphate in patients with preserved renal function and phosphate within normal physiological limits (2.5-4.5 mg/dL). Observational studies show a 35% higher risk of aortic stenosis per mg/dL increase in serum phosphate—a graded effect within the physiological range of values, suggesting that lowering phosphate may be useful in prevention. In some embodiments, sevelamer lowers serum phosphate in patients with aortic stenosis whose phosphate levels are within the physiological range.

In addition to binding dietary phosphate, sevelamer binds other substances in the gut (e.g. bile acids, bacterial endotoxins, and advanced glycation endproducts [AGEs]), leading to pleiotropic effects specific to a polymeric phosphate binder. The binding of bile acids is a recognised method for lowering serum cholesterol. Sevelamer has been found to reduce serum total cholesterol and LDL cholesterol by 15-30%, which would be expected to reduce the risk of heart attack and stroke by about 30-50%. Thus, the use of sevelamer for preventing progression of aortic stenosis carries an additional benefit of reducing serum cholesterol.

In some embodiments, sevelamer or a pharmaceutically acceptable derivative, mixture or salt thereof reduces serum total cholesterol and LDL cholesterol. The sevelamer or a pharmaceutically acceptable derivative, mixture or salt thereof may reduce the risk of heart attack and/or stroke.

Sevelamer is not systemically absorbed, resulting in few serious adverse effects. It avoids calcium toxicity associated with calcium-acetate and is the only generic non-calcium phosphate binder available, reducing cost and widening access to treatment/prevention of aortic stenosis.

The use of sevelamer in the prevention of aortic stenosis applies existing technology for a new clinical indication—repurposing a drug used in the prevention of bone disease among patients with renal failure, for the prevention of aortic stenosis in the general population. The research is timely because the prevalence of aortic stenosis is expected to double in the next few decades as the population ages, there is no known means of prevention, and generic sevelamer became available within the last year, opening avenues for an affordable means of prevention if evidence of benefit can be shown. The annual cost of generic sevelamer (800 mg three times daily) is about £166, or £1,660 over 10 years—this is less than 10% of the cost of aortic valve surgery. The cost at scale would therefore not be prohibitive and compares favourably to the alternative surgical option.

Previous work has focused on the use of sevelamer to aid the body when the body's homeostatic system had broken down to the extent that circulatory levels of phosphate ions were out of the normal, i.e. at non-physiological ranges of phosphate. However, the present inventors have made the surprising discovery that sevelamer is capable of modifying the levels of calcium and the phosphate when the body's homeostatic systems—which are quite robust in their activity—are still maintaining these ions within their normal physiological ranges.

The long latent period between mild and severe aortic stenosis provides a window of opportunity to prevent progression. By attenuating or arresting the progression of calcium phosphate deposition, sevelamer may provide substantial benefit to affected individuals. The availability of preventative treatment, also raise the possibility of screening for the aortic stenosis with a view to prevention at a population level.

The term “therapeutically effective amount” refers to an amount effective to achieve a desired and/or beneficial effect. An effective amount can be administered in one or more administrations.

### Pharmaceutically Acceptable Derivative, Mixture or Salt Thereof

The term “pharmaceutically acceptable” refers to a composition suitable for use in treatment of humans and/or animals. Typically, the formulations are relatively non-toxic and do not cause additional side effects compared to the drug delivered. In some embodiments, the pharmaceutically acceptable salt of sevelamer salt is sevelamer hydrochloride or sevelamer carbonate.

### Administration

In some embodiments, sevelamer or a pharmaceutically acceptable derivative, mixture or salt thereof is administered orally, for example with meals. When taken with meals, sevelamer binds to dietary phosphate and prevents its absorption. Sevelamer or a pharmaceutically acceptable derivative, mixture or salt thereof may be in the form of a tablet. Sevelamer tablets may be stored in blister packs or bottles.

In some embodiments, 800 mg of sevelamer or a pharmaceutically acceptable derivative, mixture or salt thereof is administered three times daily. For example, one 800 mg sevelamer tablet may be administered with each meal. Alternatively, two or three 800 mg sevelamer tablets may be administered with each meal.

In some embodiments, about 1-14 g, about 2-8 g, or about 5-6 g of sevelamer or a pharmaceutically acceptable derivative, mixture or salt thereof is administered per day. In some embodiments, about 1 g, 2 g, 3 g, 4 g, 5 g, 6 g, 7 g, or 8 g of sevelamer or a pharmaceutically acceptable derivative, mixture or salt thereof is administered per day.

### Treatment and Prevention

The treatment of aortic stenosis encompasses achieving any desirable effect, such as the ability to palliate, ameliorate, stabilize, reverse, attenuate, arrest, prevent, slow or delay the progression of the disease (e.g. aortic stenosis), increase the quality of life, and/or to prolong life. Such achievement can be measured by any method known in the art, such as measuring patient life or, in the context of aortic stenosis, measuring the extent of valve leaflet thickening and calcification via echocardiography or CT scanning. The above outcomes may be achieved by attenuating or arresting the rate of calcium phosphate deposition in a patient suffering from aortic stenosis.

The term “preventing” or “prevention” encompasses prevention of the disease, prevention of aggravation of symptoms and prevention of development of the disease.

### Aortic Stenosis

Aortic stenosis may involve narrowing of the aortic valve opening, which restricts the blood flow from the left ventricle to the aorta. Aortic stenosis is most commonly caused by age-related progressive calcification (>50% of cases) with a mean age of 65 to 70 years. Another major cause of aortic stenosis is the calcification of a congenital bicuspid aortic valve.

In some embodiments, the aortic stenosis comprises or consists of calcific aortic stenosis.

### Patients and Subjects

A “patient” encompasses an individual under surgical or medical treatment or supervision, including those individuals suffering from aortic stenosis and persons suspected of having or predisposed to have aortic stenosis. In some embodiments, the patient is a mammal. In some embodiments, the patient is a human.

A “subject” encompasses an individual who may or may not have aortic stenosis or who may be predisposed to have aortic stenosis. In some embodiments, the subject is a mammal. In some embodiments, the subject is a human.

Serum phosphate levels are homeostatically regulated by balancing dietary phosphate intake with renal excretion and bone absorption. This homeostasis is lost in patients with advanced renal disease, in whom high serum phosphate leads to secondary hyperparathyroidism, bone demineralisation and fractures.

In some embodiments, the patient does not have hyperphosphatemia. In other words, the patient's serum phosphate levels may be in the normal physiological range, i.e. between 2.5 mg/dL and 4.5 mg/dL. In some embodiments, the patient does not have chronic kidney disease or renal failure/disease. In other words, the patient may have preserved renal function. In some embodiments, the patient is not undergoing hemodialysis. In some embodiments, the patient has been identified as having aortic stenosis or being at risk of developing aortic stenosis.

The following examples are provided to illustrate but not limit the invention.

### REFERENCE EXAMPLE 1

Observational studies (Table 1) demonstrated a graded association between the progression of aortic stenosis and serum phosphate levels, within the physiological range. In the Cardiovascular Health Study (1,938 healthy people with echocardiographic measurements), there was a 40% increase in the risk of echocardiographically assessed aortic sclerosis (a mild form of aortic stenosis with valve leaflet thickening and calcification without obstruction) per mg/dL increase in serum phosphate within the physiological range of values (2.5-4.5 mg/dL), after adjustment for confounding variables, including kidney function [2]. A similar 34% increased risk of CT-detected aortic valve calcification per mg/dL increase in serum phosphate was observed in the MESA cohort study [3].

TABLE 1

| | Number | Assessment | | Serum | Relative risk per | Relative Risk per |
|---|---|---|---|---|---|---|
| | of | of aortic | Adjusted | phosphate | quartile of serum | 1.0 mg/dL increase |
| Study | subjects | valve | factors | mg/dL | phosphate (95% CI) | in serum phosphate |
| Cardiovascular | 1938 | Echo | Age, sex, race, | <3.0 | 1.0 | 1.4 (1.2-1.6) |
| Health Study | | | renal function, BP, | 3.1-3.5 | 1.2 (0.9-1.7) | |
| JACC 2011 | | | diabetes, smoking, | 3.6-4.0 | 1.5 (1.1-2.0) | |
| | | | lipids, calcium | >4.0 | 1.8 (1.2-2.6) | |
| MESA Study | 6812 | CT | Age, sex, renal | <3.0 | 1.0 | 1.34 (1.2-1.5) |
| Atherosclerosis | | | function, BP, | 3.1-3.5 | 1.1 (0.9-1.4) | |
| 2014 | | | diabetes, smoking, | 3.6-4.0 | 1.2 (0.9-1.4) | |
| | | | lipids, calcium | >4.0 | 1.6 (1.3-2.0) | |

Natural history studies showed that about 60% of patients with mild aortic stenosis progress to death or valve replacement within 5 years [1]. There were no comparable associations with serum levels of calcium. If the association with serum phosphate is causal and reversible, lowering serum phosphate by 1 mg/dL would be expected to reduce risk by about 26% (1/1.35).

Scanning electron micrographs of aortic valve tissue revealed the micro-architecture of aortic stenosis to consist of spherical particles, whose number and density increased with increasing severity of aortic stenosis. X ray diffraction methods identified the particle as a highly crystalline form of calcium phosphate, distinct from bone mineral. Particles were identical at all stages of aortic stenosis development, from mild disease (Aortic peak velocity 2.0-3.0 m/s), moderate disease (Aortic peak velocity 3.1-4.0 m/s) and severe disease (Aortic peak velocity >4.0 m/s), suggesting a common causal deposit that may be amenable to prevention [4]. The results of preclinical (in vitro) studies showed that the tendency for such crystals to form was sensitive to changes in serum phosphate, within the physiological range. In human aorta vascular smooth muscle, calcification approximately doubled with every mg/dL increase in serum phosphate, and was more pronounced with changes in serum phosphate than serum calcium [5,6].

### EXAMPLE 2

A randomised trial is conducted to determine the efficacy of sevelamer in lowering serum phosphate in patients with aortic stenosis and preventing the progression of the disease, with a view to its routine use in clinical practice for patient benefit. A double-blind randomised placebo controlled pilot cross-over trial is conducted (6 weeks taking sevelamer 2.4 g/day, 6 weeks taking sevelamer 7.2 g/day and 6 weeks taking placebo, allocated in random order).

The cross-over design (in which each participant is their own control) provides the statistical power to assess efficacy in lowering phosphate with few patients and the 2 doses allow the trade-off between efficacy and tolerability to be assessed. The cross-over design was chosen over a parallel group design because it minimises between person-variation in response to treatment, and allows clinically important treatment effects to be tested with high power, using a small number of patients. It is therefore an extremely efficient and cost-effective study design. This pilot study also develops the recruitment pathways for a definitive trial.

Study participants: Patients with mild to moderate aortic stenosis, defined as a trans-aortic valve velocity of 2.0-4 m/s, are enrolled because they are the group who stand to benefit from prevention, are therefore likely to be motivated to participate, and have an early stage of the disease that is unlikely to require surgical intervention during the course of the trial.

Patients are excluded if: 1) they have severe aortic stenosis 2) they require phosphate binding drugs for other reasons, 3) are taking drugs that may interact with phosphate binding drugs, or 4) have a history of bowel obstruction (a specific contraindication).

Feasibility Study: A feasibility study was conducted over 3 months at St Barts Hospital and showed that, on average, 12 eligible patients with mild-moderate aortic stenosis (mean aortic velocity 2.3 m/s, S.D 0.5) were seen in one echocardiography clinic each month. With 6 clinics running at St Barts and St Thomas's Hospitals (72 eligible patients/month), we have the opportunity to recruit the required number of patients in to the study. Since the outline application, we have continued to monitor echocardiography clinics at St Barts Hospital and have built up a list of 90 potentially eligible patients who can be contacted and invited to participate in the study. We have also sought the views of 20 consecutive patients with mild-moderate aortic stenosis, being reviewed in one cardiology clinic. Of the 20 patients asked, 16 said they would be willing to take part in the study. These patients represent the end-users of this technology, and indicate a high level of engagement with the project plan.

Primary study endpoint: Within-person placebo subtracted differences in serum phosphate after each treatment period.

Acceptability endpoint: Adherence (by pill-count) and side-effects (questionnaire) after each treatment period.

Dose: Typical doses of sevelamer used in patients with renal disease are 2.4 g/day to 7.2 g/day. The average dose taken in a trial by Kettler et al, [7] to determine the effect of lowering serum phosphate in patients with end stage renal failure was 5.5 g/day (dose range 4.8 g/day to 7.2 g/day). In the trial, within person comparisons of 2.4 g/day and 7.2 g/day with placebo provides a sufficiently wide contrast in dose exposure, within the range used in clinical practice, to determine a difference in dose-response, if there is one, and to assess acceptability. This is important, because sevelamer is known to have gastro-intestinal side-effects (such as a sense of fullness after eating, nausea and constipation), which whilst not serious, are likely to be dose-related and could lead to non-adherence. The pilot trial therefore provides an important opportunity to examine the trade-off between efficacy and acceptability between the two doses. If a lower dose is more acceptable and provides sufficient efficacy it would have practical advantages to its use.

Sevelamer, at low dose (2.4 g/day) and high dose (7.2 g/day) is compared with placebo in 72 patients with aortic stenosis. A trial of 72 patients has 80% power to show a mean reduction in serum phosphate of at least 0.5 mg/dL, comparing each dose of sevelamer (2.4 g/day and 7.2 g/day) to placebo and comparing the higher versus the lower dose, at the 5% level of significance. The sample size allows for a 20% non-completion rate.

Anticipated effect size. The average reduction in serum phosphate using sevelamer (5 g/day) was 1.4 mg/dL (Standard Deviation 1.0) in the trial by Kettler et al, in patients with end stage renal failure. To be conservative and allow for the lower starting serum phosphate levels in patients without renal failure, we predict a smaller reduction in serum phosphate (1.0 mg/dL at the higher dose and 0.5 mg/dL at the lower dose) and a larger SD (1.3 mg/dL).

Washout: The trial by Kettler et al. showed that serum phosphate levels returned to baseline (i.e. the effect of sevelamer washed out) within 2 weeks of stopping treatment and that the full reduction of serum phosphate was achieved within 4 weeks [7]. The 6 week treatment period is therefore long enough for the drug-effect from the previous period to have cleared well before the end of the next treatment period, obviating the need for a separate washout phase. This simplifies the trial design and avoids unnecessary extra visits for participating patients.

Medication adherence: Failure to take medication as prescribed in clinical trials reduces the pharmacological assessment of efficacy. We aim to maximise adherence in the pilot trial using an automated text-message software application which we developed at the Wolfson Institute of Preventive Medicine and have shown, in a randomised trial [8], improves adherence to prescribed preventive cardiovascular medications; 25% (38/150 patients) in the control group (no text messages) stopped medication completely or took it less than directed versus 9% (14/1501) in the text message group, after 6 months of treatment. In addition to reminding patients to take their medication at specified times, patients were required to send a text message reply indicating whether or not medication had been taken, enabling patients who had not taken their medication to be contacted and offered help. Patients who have a mobile phone would be eligible for inclusion in the study and all would receive daily text-messages in this way. Over 90% of people now own a mobile phone (OfCom 2014), so this would lead to few exclusions.

Analysis plan: The results are analysed at the end of the 18-week crossover period. Participants who do not complete the trial are excluded from the analysis without introducing bias, because of the cross-over design. Within-person placebo-subtracted differences in serum phosphate at the end of each treatment period are calculated to give the reductions in serum phosphate on 2.4 g/day, on 7.4 g/day and the difference between these two doses. The average of these respective within-person differences, are estimated across all patients. The placebo-adjusted prevalence of side-effects (e.g. reports of nausea, vomiting, constipation) at each dose is also be compared.

Synthon is providing 36,288 (800 mg) tablets of sevelamer and 45,360 tablets of matching placebo. Sevelamer has an EU/MIHRA marketing authorisation which means their summary of medicinal product characteristics is sufficient for clinical trial authorisation. Sharp Clinical Services receive the sevelamer and placebo, carry out identity testing, package in to blister strips and cartons according to a randomisation schedule, apply approved labels and issue QP release for use in the trial.

Synthon have undertaken pilot work to develop the matching placebo, which took 4 months and considerable expertise because over-encapsulation was not possible given the size of the tablet. The packaging, labeling and QP release activities that would be carried out by Sharp are mandatory regulatory requirements for a randomised trial.

### EXAMPLE 3

The Target Product Profile for sevelamer (see Table 2) provides an indication of how the drug would be used, its safety, dosing, delivery and pricing. Given that the drug is already available and licensed for use in patients with renal disease and has been used in practice for about 20 years, we can be reasonably confident that this is a realistic profile for its use in the prevention of aortic stenosis.

TABLE 2

| Attribute | Desired | Acceptable |
|---|---|---|
| Mechanism of Action* | Dietary phosphate - binding and | Dietary phosphate - binding and |
| | serum phosphate lowering | serum phosphate lowering |
| Efficacy & product benefit | Effective in arresting the | Effective in slowing the |
| | progression of aortic stenosis | progression of aortic stenosis |
| Safety & Tolerability | <0.01% serious adverse events | 0.1% serious adverse events |
| | <10% mild adverse effects | <20% mild adverse effects |
| | No monitoring for adverse events | Periodic clinical and biochemical |
| | | monitoring |
| Dosing/administration/regimen | One 800 mg tablet with each meal | Three tablets with each meal |
| | (ie 2.4 g/day) | (ie 7.2 g/day) |
| Delivery system, product* | Tablet in blister strip or bottle, | Tablet in blister strip or bottle |
| presentation | with no special storage conditions | with no special storage conditions |
| Market | All patients with mild- | 40% of patients with mild- |
| | moderate aortic stenosis | moderate aortic stenosis |
| Pricing (generic prescribing) | £210 per patient per year. | £630 per patient per year. |

*Desired = acceptable because the drug (sevelamer) is available so the properties are known

### REFERENCES

1. Rosenhek R, Klaar U, Schemper M, Scholten C, Heger M, Gabriel H, Binder T, Maurer G, Baumgartner H. Mild and moderate aortic stenosis. Eur Heart J 2004; 25:199-205. doi:10.1016/j.ehj.2003.12.002.

2. Linefsky J P, O'Brien K D, Katz R, de Boer I H, Barasch E, Jenny N S, Siscovick D S, Kestenbaum B. Association of Serum Phosphate Levels With Aortic Valve Sclerosis and Annular Calcification. J Am Coll Cardiol 2011; 58(3):291-297. doi:10.1016/j.jacc.2010.11.073.

3. Linefsky J P, O'Brien K D, Sachs M, Katz R, Eng J, Michos E D, Budoff M J, de Boer I, Kestenbaum B. Serum phosphate is associated with aortic valve calcification in the Multi-ethnic Study of Atherosclerosis (MESA). Atherosclerosis 2014; 233:331-337. doi:10.1016/j.atherosclerosis.2013.12.051.

4. Bertazzo S, Gentleman E, Cloyd K L, Chester A H, Yacoub M H, Stevens M M. Nano-analytical electron microscopy reveals fundamental insights into human cardiovascular tissue calcification. Nat Mater 2013; 12:576-583. doi:10.1038/nmat3627.

5. Jono S, McKee M D, Murry C E, Shioi A, Nishizawa Y, Mori K, Morii H, Giachelli C M. Phosphate Regulation of Vascular Smooth Muscle Cell Calcification. Circ Res 2000; 87:e10-e17. doi:10.1161/01.RES.87.7.e10.

6. Yang H, Curinga G, Giachelli C M. Elevated extracellular calcium levels induce smooth muscle cell matrix mineralization in vitro. Kidney Int 2004; 66:2293-2299.

7. Ketteler M, Rix M, Fan S, Pritchard N, Oestergaard O, Chasan-Taber S, Heaton J, Duggal A, Kalra P A. Efficacy and Tolerability of Sevelamer Carbonate in Hperphosphatemic Patients Who Have Chronic Kidney Disease and Are Not on Dialysis. Clin J Am Soc Nephrol 2008; 3(4):1125-1130. doi:10.2215/CJN.05161107.

8. Wald D S, Bestwick J P, Raiman L, Brendell R, Wald N J. Randomised trial of text messaging on adherence to cardiovascular preventive treatment (INTERACT trial). PLoS ONE 2014; 9(12):e114268. doi:10.1371/journal.pone.0114268.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

Although the foregoing invention has been described in some detail by way of illustration and example for purposes of clarity of understanding, it will be apparent to those skilled in the art that certain minor changes and modifications will be practised. Therefore, the description and examples should not be construed as limiting the scope of the invention, which is delineated by the appended claims.

1. A method of treating and/or preventing aortic stenosis in a patient in need thereof, said method comprising administering to the patient a therapeutically effective amount of sevelamer or a pharmaceutically acceptable derivative, mixture or salt thereof.

2. The method of claim 1, wherein said method attenuates or arrests the deposition of calcium phosphate crystals on the aortic valve of the patient.

3. The method of claim 1, wherein the patient does not have hyperphosphatemia.

4. The method of claim 1, wherein the patient's serum phosphate level is from 2.5 mg/dL to 4.5 mg/dL.

5. The method of claim 1, wherein the patient does not have chronic kidney disease.

6. The method of claim 1, wherein the sevelamer salt is sevelamer hydrochloride or sevelamer carbonate.

7. The method of claim 1, wherein the sevelamer or a pharmaceutically acceptable derivative, mixture or salt thereof is administered orally, preferably with meals.

8. The method of claim 1, wherein 800 mg of sevelamer is administered three times daily.

9. The method of claim 1, wherein:
(a) about 1-14 g of sevelamer is administered per day;
(b) about 2-8 g of sevelamer is administered per day; or
(c) about 5-6 g of sevelamer is administered per day.

10. The method of claim 1, wherein: about 2 g, about 3 g, about 4 g, about 5 g, about 6 g, about 7 g, or about 8 g of sevelamer is administered per day.

11. The method of claim 1, wherein the sevelamer or a pharmaceutically acceptable derivative, mixture or salt thereof is co-administered with a bisphosphonate.

12. The method of claim 1, wherein the patient has been identified as having aortic stenosis or being at risk of developing aortic stenosis.

13. The method of claim 1, wherein the sevelamer or a pharmaceutically acceptable derivative, mixture or salt thereof reduces serum total cholesterol and LDL cholesterol.

14. The method of claim 1, wherein the sevelamer or a pharmaceutically acceptable derivative, mixture or salt thereof reduces the risk of heart attack and/or stroke.

15. A method of identifying a subject that would benefit from treatment with sevelamer or a pharmaceutically acceptable derivative, mixture or salt thereof, said method comprising:
(a) determining whether the subject shows a symptom of aortic stenosis; and
(b) where the subject shows a symptom of aortic stenosis, making a record that the subject would benefit from treatment with sevelamer or a pharmaceutically acceptable derivative, mixture or salt thereof.

16. The method of claim 15, wherein the determining in step (a) involves echocardiography and/or a Computed Tomography (CT) scan.

17. The method of claim 15, wherein the method further comprises determining that the subject does not have hyperphosphatemia.

18. A method of treating and/or preventing aortic stenosis in a patient comprising:
(a) determining whether the patient has a symptom of aortic stenosis; and
(b) where the subject shows a symptom of aortic stenosis, administering to the patient an effective amount of sevelamer or a pharmaceutically acceptable derivative, mixture or salt thereof.

19. The method of claim 18, wherein the screening in step (a) involves echocardiography and/or a Computed Tomography (CT) scan.

20. The method of claim 18, wherein the method further comprises determining that the subject does not have hyperphosphatemia.
